(19) United States
(12) Patent Application Publication
(10) Pub. No.: US 2024/0110854 A1
(43) Pub. Date: Apr. 4, 2024

(54) SYSTEMS AND METHODS FOR PROCESSING STOOL SAMPLES

(71) Applicant: BiomeSense, Inc., Chicago, IL (US)

(72) Inventors: Dylan Nichols, Chicago, IL (US); Kevin Honaker, Chicago, IL (US)

(21) Appl. No.: 18/328,429

(22) Filed: Jun. 2, 2023

Related U.S. Application Data
(63) Continuation of application No. PCT/US2021/063379, filed on Dec. 14, 2021.
(60) Provisional application No. 63/125,816, filed on Dec. 15, 2020.

(51) Int. Cl. G01N 1/38; G01N 1/40

(52) U.S. Cl. CPC G01N 1/38; G01N 1/4044; A61B 10/0038

(57) ABSTRACT

The present disclosure provides methods and systems directed to providing and processing stool samples, including processing substrates comprising stool samples. A stool sample may be collected from a subject using a substrate. The substrate and/or stool sample may be further processed. One or more biomolecules may be extracted from the stool sample.

### CROSS REFERENCE

This application is a continuation of International Application No. PCT/US2021/063379, filed on Dec. 14, 2021, which claims the benefit of U.S. Provisional Application No. 63/125,816, filed on Dec. 15, 2020, which is herein incorporated by reference in its entirety.

### BACKGROUND

A sample may be collected for various purposes, such as identification of a property of the sample. The sample may be a biological sample or biology-derived sample. Biological samples may be processed, such as for disease detection and diagnosis, identification of contaminants, characterization of contents. Various approaches for processing samples may be performed, such as polymerase chain reaction (PCR) and nucleic acid sequencing.

Biological samples may be collected using a variety of approaches, such as physical capture. A substrate may be used to collect the biological sample. Devices or systems may be employed to capture or collect the substrate and/or biological sample and enable further analysis of the biological sample.

Biological samples may be subjected to various processes, such as chemical or physical processes. Samples may be subjected to heating or cooling, chemical reactions, mechanical processes, such as to yield a sample or species that can be processed qualitatively or quantitatively.

### SUMMARY

In an aspect, provided herein is a method for processing a stool sample, comprising: (a) providing (i) a substrate comprising the stool sample and (ii) a liquid; (b) processing the substrate, thereby generating a mixture comprising the liquid and portions of the substrate; and (c) extracting at least a portion of the stool sample from the mixture, thereby generating a processed stool sample.

In some embodiments, (b) or (c) is performed automatically. In some embodiments, (b) and (c) are performed automatically.

In some embodiments, (b) comprises mixing the substrate and the liquid. In some embodiments, the mixing is performed using a mechanical force. In some embodiments, the mixing is performed using a blade or propeller.

In some embodiments, the liquid comprises water or a buffer.

In some embodiments, (a) occurs in a vessel. In some embodiments, the vessel is coupled to a toilet. In some embodiments, the vessel is a part of a system that is configured to process the mixture or the processed stool sample. In some embodiments, the system further comprises a lysis unit configured to lyse a cell in the processed stool sample. In some embodiments, the system comprises a storage unit configured to store the processed stool sample or a biomolecule derived from the processed stool sample. In some embodiments, the method further comprises, subsequent to (c), cleaning the vessel. In some embodiments, the cleaning is performed automatically.

In some embodiments, the at least the portion of the stool sample comprises a biomolecule. In some embodiments, the biomolecule comprises one or more members selected from the group consisting of: a cell, an organelle, a protein, a peptide, a lipid, a carbohydrate, and a nucleic acid molecule.

In some embodiments, the substrate is made of a pliable material. In some embodiments, the pliable material is toilet paper.

In some embodiments, the method further comprises processing the processed stool sample. In some embodiments, the processing comprises extracting one or more biomolecules from the processed stool sample. In some embodiments, the one or more biomolecules comprises one or more members selected from the group consisting of: a cell, an organelle, a protein, a peptide, a lipid, a carbohydrate, and a nucleic acid molecule. In some embodiments, the nucleic acid molecule is deoxyribonucleic acid (DNA). In some embodiments, the cell is a bacterial cell, a fungal cell, or a human cell.

In another aspect, disclosed herein is a method for processing a stool sample, comprising: (a) providing a substrate comprising the stool sample; (b) agitating the substrate to yield a plurality of portions of the substrate, wherein a portion of the plurality of portions of the substrate comprises at least a portion of the stool sample; and (c) extracting a biomolecule from the at least the portion of the stool sample of (b).

In some embodiments, (b) or (c) is performed automatically. In some embodiments, (b) and (c) are performed automatically.

In some embodiments, (b) comprises mixing the substrate and a liquid. In some embodiments, the mixing is performed using a mechanical force. In some embodiments, the mixing is performed using a blade or propeller. In some embodiments, the liquid comprises water or a buffer.

In some embodiments, (a) occurs in a vessel. In some embodiments, the vessel is coupled to a toilet. In some embodiments, the vessel is a part of a system that is configured to process the portion. In some embodiments, the system further comprises a lysis unit configured to lyse a cell in the portion. In some embodiments, the system comprises a storage unit configured to store the biomolecule. In some embodiments, the method further comprises, subsequent to (c), cleaning the vessel. In some embodiments, the cleaning is performed automatically.

In some embodiments, the at least the portion of the stool sample comprises the biomolecule. In some embodiments, the biomolecule comprises one or more members selected from the group consisting of: a cell, an organelle, a protein, a peptide, a lipid, a carbohydrate, and a nucleic acid molecule.

In some embodiments, the substrate is made of a pliable material. In some embodiments, the pliable material is toilet paper.

In some embodiments, the biomolecule comprises one or more members selected from the group consisting of: a cell, an organelle, a protein, a peptide, a lipid, a carbohydrate, and a nucleic acid molecule. In some embodiments, the nucleic acid molecule is deoxyribonucleic acid (DNA). In some embodiments, the cell is a bacterial cell, a fungal cell, or a human cell.

In another aspect, provided herein is a system for processing a stool sample provided on a substrate, comprising: a vessel configured to process (i) the stool sample provided on the substrate and (ii) a liquid, to generate a mixture comprising the liquid and portions of the substrate; and an extraction unit configured to extract at least a portion of the stool sample from the mixture to generate a processed stool sample.

In some embodiments, the extraction unit comprises a lysis unit configured to lyse one or more cells present in the mixture to generate a lysed product. In some embodiments, the extraction unit further comprises a storage unit configured to extract the at least the portion of the sample from the lysed product. In some embodiments, the storage unit is in fluidic communication with a reservoir comprising reagents for extracting a biomolecule from the at least the portion of the sample. In some embodiments, the biomolecule is a cell, an organelle, a protein, a peptide, a lipid, a carbohydrate, or a nucleic acid molecule. In some embodiments, the storage unit comprises a support for capturing a biomolecule from the at least the portion of the sample. In some embodiments, the support is a bead. In some embodiments, the bead is a magnetic bead.

In some embodiments, the system further comprises a waste unit configured to store and/or expel waste from the vessel or the extraction unit.

In some embodiments, the system further comprises a storage unit configured to store a biomolecule extracted from the processed stool sample. In some embodiments, the storage unit comprises a moveable stage configured to couple to a sample container. In some embodiments, the sample container is disposable.

In some embodiments, the system further comprises a reservoir in fluidic communication with the extraction unit.

In some embodiments, the system further comprises a metering unit configured to meter volumes of liquids provided to the extraction unit and/or the vessel.

In some embodiments, the system further comprises a power source. In some embodiments, the power source comprises a battery.

In some embodiments, the vessel comprises a chamber for processing the stool sample and the liquid.

In some embodiments, the vessel comprises a blade, propeller, or blender for mixing, agitating, or homogenizing the stool sample and the liquid.

In some embodiments, the extraction unit comprises a storage unit, wherein the storage unit is configured to couple to a sample container. In some embodiments, the sample container is a multi-well plate.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 schematically illustrates an example workflow for providing and processing a substrate comprising a stool sample.

FIG. 2 schematically illustrates an example of a vessel that is configured to receive and process a substrate comprising a stool sample.

FIG. 3 schematically shows an example of a vessel.

FIG. 4 schematically shows another example of a vessel.

FIGS. 5A-5B schematically illustrate example vessels. FIG. 5A shows a vessel in a horizontal configuration. FIG. 5B shows a vessel in a vertical configuration.

FIG. 6 shows different perspective views (front, top, and side) of another example vessel in a horizontal configuration.

FIG. 7 shows an exploded view of the example vessel of FIG. 6.

FIG. 8 schematically illustrates a view of a component of the vessel comprising a blade.

FIG. 9 shows another view of an example vessel in a horizontal configuration.

FIG. 10 schematically illustrates a portion of a system described herein comprising a vessel.

FIG. 11A schematically illustrates an example system described herein. FIG. 11B schematically illustrates another example system described herein.

FIG. 12 shows an example of a user interface of the system.

FIG. 13 illustrates a housing of an example system described herein.

FIG. 14 shows a computer system that is programmed or otherwise configured to implement methods provided herein.

FIG. 15 schematically shows another example vessel in a vertical configuration.

FIG. 16 schematically shows an example of a lysis unit.

FIG. 17 schematically shows an example storage unit.

FIG. 18 schematically shows an example cartridge of a storage unit described herein.

FIG. 19 schematically shows an example waste unit.

FIG. 20 schematically shows a fluid metering system.

FIG. 21 shows example data obtained from using the systems and methods described herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The term “subject,” as used herein, generally refers to an animal, such as a mammal (e.g., human) or avian (e.g., bird), or other organism, such as a plant. For example, the subject can be a vertebrate, a mammal, a rodent (e.g., a mouse), a primate, a simian, or a human. Animals may include, but are not limited to, farm animals, sport animals, and pets. A subject can be a healthy or asymptomatic individual, an individual that has or is suspected of having a disease (e.g., cancer), or a pre-disposition to the disease, an infection, and/or an individual that is in need of therapy or suspected of needing therapy. A subject can be a patient. A subject can be a microorganism or microbe (e.g., bacteria, fungi, archaea, viruses).

The term “sequencing,” as used herein, generally refers to methods and technologies for determining the sequence of nucleotide bases in one or more polynucleotides. The polynucleotides can be, for example, nucleic acid molecules such as deoxyribonucleic acid (DNA) or ribonucleic acid (RNA), including variants or derivatives thereof (e.g., single stranded DNA, complementary DNA, etc.). Sequencing can be performed by various systems currently available, such as, without limitation, a sequencing system by Illumina®, Pacific Biosciences (PacBio®), Oxford Nanopore®, or Life Technologies (Ion Torrent®). Alternatively or in addition to, sequencing may be performed using nucleic acid amplification, polymerase chain reaction (PCR) (e.g., digital PCR, quantitative PCR, or real time PCR), or isothermal amplification. Such systems may provide a plurality of raw genetic data corresponding to the genetic information of a subject (e.g., human), as generated by the systems from a sample provided by the subject. In some examples, such systems provide sequencing reads (also “reads” herein). A read may include a string of nucleic acid bases corresponding to a sequence of a nucleic acid molecule that has been sequenced. In some situations, systems and methods provided herein may be used with proteomic information.

The term “sample,” as used herein, generally refers to a biological sample of a subject. The biological sample may comprise any number of macromolecules, for example, cellular macromolecules. The sample may be a stool sample. The sample may be a cell line or cell culture sample. The sample can include one or more cells. The sample can include one or more microbes. The biological sample may be a nucleic acid sample or protein sample. The biological sample may also be a carbohydrate sample or a lipid sample. The biological sample may be derived from (e.g., processed from) another sample. The sample may be a stool sample or derived from a stool sample. The sample may be a tissue sample, such as a biopsy, core biopsy, needle aspirate, or fine needle aspirate. The sample may be a fluid sample, such as a blood sample, urine sample, or saliva sample. The sample may be a skin sample. The sample may be a cheek swab. The sample may be a plasma or serum sample. The sample may be a cell-free or cell free sample. A cell-free sample may include extracellular polynucleotides. Extracellular polynucleotides may be isolated from a bodily sample that may be selected from the group consisting of blood, plasma, serum, urine, saliva, mucosal excretions, sputum, stool, and tears.

### Processing of Substrates Comprising Stool Samples

In an aspect, the present disclosure provides methods and systems for processing one or more stool samples. The methods and systems herein may involve provision of a stool sample, which may be coupled to or comprised within a substrate, and processing the stool sample and/or substrate. A method for processing a stool sample may comprise (a) providing: (i) a substrate comprising a stool sample and (ii) a liquid; (b) processing the substrate, thereby generating a mixture comprising the liquid and portions of the substrate; and (c) extracting at least a portion of the stool sample from the mixture, thereby generating a processed stool sample. The methods and systems disclosed herein may be automated or partially automated and may be useful in minimizing a subject's interaction with a stool sample as well as provide facile processing (e.g., biomolecule extraction, such as DNA extraction) of stool samples. Such methods and systems may be applicable in a variety of settings, e.g. for home, commercial, medical, clinical, or laboratory purposes.

The methods and systems disclosed herein may be useful in extracting stool or a derivative thereof (e.g., a portion of stool, a processed stool sample, a biomolecule from the stool) from a substrate comprising a stool sample. The substrate may comprise or be made of, for instance, a pliable material, such as toilet paper or tissue paper. The substrate may have any useful material properties, such as a designated porosity, absorbance, dissolvability, or degradability in a liquid (e.g., water, buffer), solubility under a defined condition (e.g., pH, salt content, temperature, etc.). The substrate may be used to collect the stool sample and subsequently be processed, using the systems and methods provided herein.

A system for processing stool samples may be configured to extract stool or derivative thereof (e.g., a biomolecule, portion of the stool sample) from a substrate comprising a stool sample. For instance, a subject may collect the stool sample using the substrate (e.g., toilet paper). The substrate comprising the stool sample may be deposited into the system, which may then process the substrate to generate a processed stool sample. Such a system may deliver a liquid (e.g., water, buffer, processing solutions) to the substrate and process the liquid and the substrate. Such processing may include, for example, mixing, agitating, homogenizing, chopping (e.g., via use of a blade or propeller), shearing, or other mechanical processes (e.g., application of mechanical force such as pressure, shear stress, etc.) that may be used to generate a mixture comprising the liquid and portions of the substrate. Further processing may be performed, e.g., extraction of a portion of the stool sample from the mixture, extraction of a biomolecule from the mixture, etc.

One or more processes described herein may be performed automatically. Automated processes may not require a subject to interact with the system in order for the operations to be performed. For example, a subject may collect a stool sample using a substrate (e.g., toilet paper). The substrate may then be deposited into the system and all processing, or a portion thereof, may be performed automatically, without human intervention. As described herein, the processing of the substrate to generate a mixture comprising a liquid and portions of the substrate (e.g., homogenization, mixing, shearing, breaking of the substrate, etc.) may be performed automatically. Alternatively or in addition to, the extraction processes (e.g., extraction of a portion of the stool sample or a biomolecule therefrom) may be performed automatically.

The substrate comprising the stool sample may be deposited (e.g., by the user, or automatically) into a vessel. The vessel may be coupled to a toilet, or the vessel may be separate from the toilet. The vessel may be a part of the system that is configured to process the substrate or the stool sample; for example, the processing of the substrate (e.g., generating the mixture comprising the liquid and portions of the substrate, extracting a portion of the stool sample or a biomolecule from the stool sample) may be performed in the vessel, and the processed substrate (e.g., mixture) may be directed elsewhere in the system for further processing. Alternatively or in addition to, the vessel may be a part of the system that processes the substrate or the processed stool sample. In such an example, the vessel may be used to collect the substrate comprising the stool sample and, in some instances, provide a liquid for processing of the stool sample. The processing of the stool sample (e.g., mixing, extraction) may then occur in the vessel or in another component of the system. In some instances, the mixture may be generated in the vessel, and further processing (e.g., extracting a portion of the stool sample or biomolecule from the stool sample) may be performed in another component of the system.

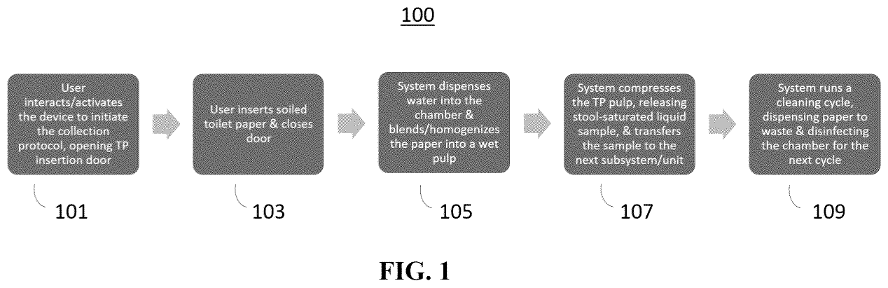

FIG. 1 provides an example workflow 100 for processing a substrate (e.g., toilet paper) comprising a stool sample. In operation 101, the system may be activated, either automatically or by a subject (e.g., a user, a patient, etc.). The system may comprise a vessel, as described herein, which may be configured to receive the substrate (e.g., toilet paper) comprising the stool sample. Next, in operation 103, the subject may deposit the substrate comprising the stool sample into the system (e.g., vessel). In operation 105, the system may dispense a liquid (e.g., water, buffers) into the vessel (e.g., chamber), and a mechanical force may be applied within the vessel. For example, the vessel may comprise a blade, blender, propeller, hose, vortex, etc., which may be used to mix, blend, homogenize or otherwise apply mechanical force on the substrate. Such processing may be used to generate a mixture comprising the liquid and portions (e.g., pieces, fibers) of the substrate. In operation 107, the system may further process the mixture. For example, the system may compress the mixture, thereby releasing or extracting a stool-saturated liquid from the mixture. The stool-saturated liquid may then be directed to a different portion of the system for further processing (e.g., lysing of cells within the stool-saturated liquid, extraction of a biomolecule, purification, storage, etc.). In operation 109, the system may be cleaned, e.g., removing waste products, disinfection or sterilization, etc. Any operation of workflow 100 may be automated. For instance, any of operations 101, 103, 105, 107, and 109 may be performed automatically (e.g., without any involvement from a user). In an example, operations 105, 107 and 109 are automated.

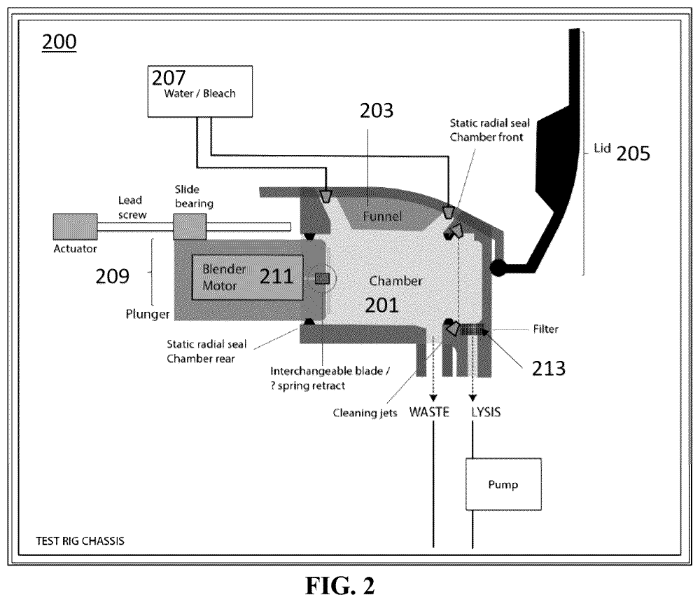

FIG. 2 schematically illustrates an example of a vessel that is configured to receive and process a substrate comprising a stool sample. The vessel 200 may comprise a chamber 201 in which the substrate and stool sample are received and processed. The vessel 200 may comprise a funnel 203, to facilitate entry of the substrate (and stool sample) into the chamber 201. In some instances, the vessel 200 comprises a lid or door 205, which may toggle between an “open” configuration in which the chamber 201 is accessible to the user and a “closed” configuration, in which the chamber is closed or sealed. In some instances, the substrate is processed in the chamber 201, and the vessel may comprise mechanical parts to facilitate such processing. For example, the vessel 200 may comprise a plunger 209 which is configured to move the contents of the chamber 201. The plunger 209 may be configured to move with respect to the chamber 201 using a variety of mechanisms, such as a screw mechanism, slide bearing, and movement actuator. Within or adjacent to the plunger 209, a motor 211 may be provided. Alternatively, or in addition to, a motor may be provided separately from the plunger 209. The motor may be used to drive movement of one or more blades or propellers, which can be used to mix or agitate the substrate within the chamber 201. The vessel may comprise fluid inlets that are connected to one or more liquid sources 207 (e.g., water, buffers, sanitizing agents such as bleach or chlorine). The vessel 200 can comprise additional elements that may be useful for sample processing, such as seals (e.g., gaskets, o-rings, rubber seals), filters, membranes, actuators, fluid inlets and outlets, etc.

In some instances, the motor 211 and coupled blades or propellers may be used to mix, agitate, homogenize, or otherwise mechanically process the substrate comprising the stool sample and the liquid (e.g., water, buffers, etc.), thereby generating a mixture comprising portions of the substrate (e.g., fibers, broken-up pieces, etc.) and the liquid. The mixture may then be directed elsewhere within or out of the chamber 201 using the plunger 209. In one non-limiting example, the plunger 209 may move the substrate, stool sample, or mixture thereof to an opposing wall of the chamber 201. Upon compression of the mixture against the opposing wall, the mixture may release a liquid comprising a portion of the stool sample (e.g., stool-saturated liquid), which may exit out of the chamber 201 via a port 213. In some instances, the port 213 comprises a filter, which may be used to filter out pieces or portions of the substrate from the mixture, thereby releasing the liquid out of the port 213. The liquid may then be directed to another portion of the system or output for further processing (e.g., in a laboratory). The vessel 200 may additionally comprise a port for transporting agents such as waste out of the chamber 201, e.g., to a different location of the system (e.g., storage unit), or out of the system to a toilet or sewage drain. The vessel 200 may subsequently be cleaned or disinfected, e.g., using a sterilization agents, e.g., bleach, chlorine, antimicrobial agents, peroxide, ammonia, alcohol, an acid, a base, minerals, soap, etc. or a combination thereof. The sterilization agents may also exit the vessel 200 via the waste port.

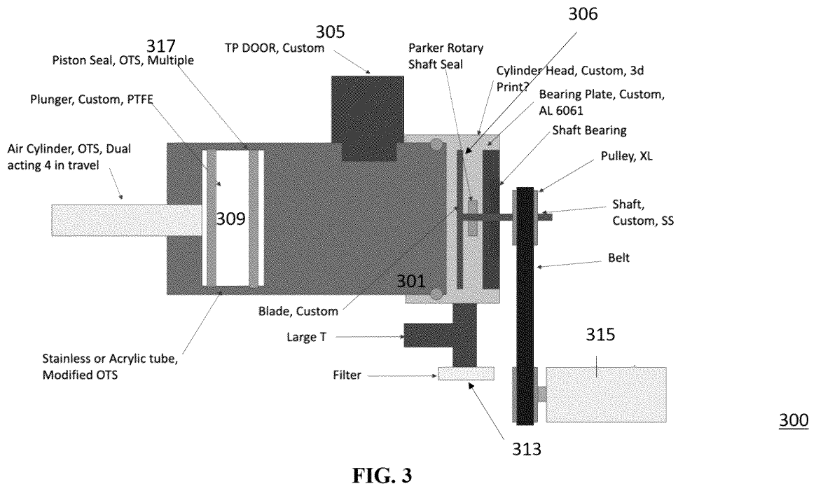

FIG. 3 schematically shows an example of a vessel, similar to that illustrated in FIG. 2, that is configured to receive and process a substrate comprising a stool sample. The vessel 300 may comprise a chamber 301 in which the substrate and stool sample are received and processed. The vessel 300 may comprise an opening that is accessible through a door 305, to facilitate entry of the substrate (and stool sample) into the chamber 301. In some instances, the substrate is processed in the chamber 301; the vessel may comprise mechanical parts to facilitate such processing. For example, the vessel 300 may comprise a plunger 309 which is configured to move the contents of the chamber 301. The plunger 309 may be configured to move with respect to the chamber 301 (shown in a horizontal direction) using a variety of mechanisms, such as a screw mechanism, slide bearing, and movement actuator. The plunger 309 may comprise an air cylinder. The plunger 309 may comprise a coating or comprise a material that is useful in preventing adsorption, e.g., teflon, polytetrafluoroethylene (PTFE), or other material. The plunger 309 may comprise gaskets, o-rings, or other seals 317. The vessel 300 may comprise one or more blades 306 or propellers, which can be used to mix or agitate the substrate within the chamber 301. The vessel 300 may comprise fluid inlets that are connected to one or more liquid sources (e.g., water, buffers, sanitizing agents such as bleach or chlorine). The vessel 300 can comprise additional elements that may be useful for sample processing, such as seals (e.g., gaskets, o-rings, rubber seals), filters (e.g., as depicted in 313), belts, membranes, actuators, fluid inlets and outlets, bearings, pulleys or other movement actuators, connection parts, motors (e.g., 315) etc. Any of the parts may be commercially available or custom-made.

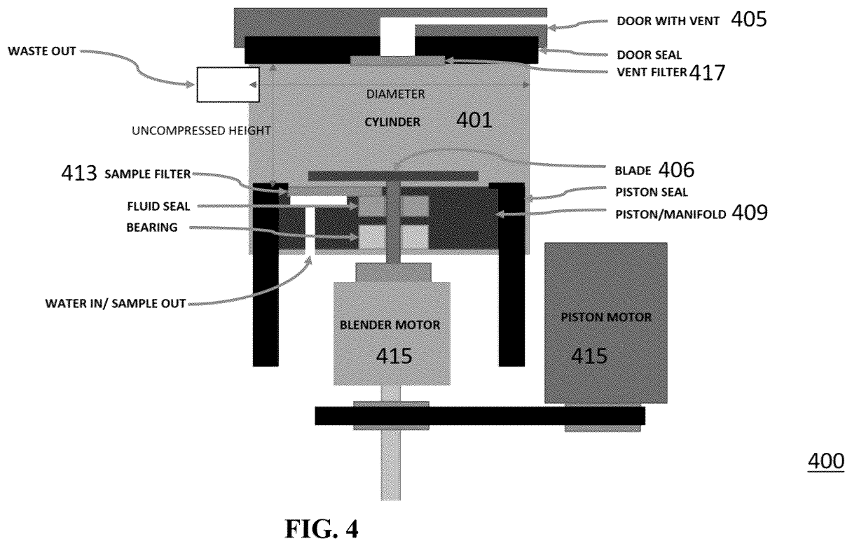

FIG. 4 schematically shows an example of a vessel, similar to that illustrated in FIG. 2 and FIG. 3, that is configured to receive and process a substrate comprising a stool sample. The vessel 400 may be oriented in a vertical construction. The vessel 400 may comprise a chamber 401 in which the substrate and stool sample are received and processed. The vessel 400 may comprise an opening that is accessible through a door 405, to facilitate entry of the substrate (and stool sample) into the chamber 401. In some instances, the substrate is processed in the chamber 401; the vessel may comprise mechanical parts to facilitate such processing. For example, the vessel 400 may comprise a piston 409 which is configured to move the contents of the chamber 401. The piston 409 may be configured to move with respect to the chamber 401 (shown in a vertical direction) using a variety of mechanisms, such as a screw mechanism, slide bearing, and movement actuator. The piston 409 may comprise a coating or comprise a material that is useful in preventing adsorption, e.g., teflon, polytetrafluoroethylene (PTFE), or other material. The piston 409 may comprise gaskets, o-rings, or other seals. The vessel 400 may comprise one or more blades 406 or propellers, which can be used to mix or agitate the substrate within the chamber 401. The vessel 400 may comprise fluid inlets that are connected to one or more liquid sources (e.g., water, buffers, sanitizing agents such as bleach or chlorine). The vessel 400 can comprise additional elements that may be useful for sample processing, such as seals (e.g., gaskets, o-rings, rubber seals), filters (e.g., outlet or sample filter 413 and door vent filter 417), membranes, actuators, fluid inlets and outlets, etc. For example, the vessel 400 may comprise a piston seal, a fluid inlet or outlet seal, a door seal, bearings, etc. One or more motors 415 may be used, e.g., for actuation of the blender or of the piston (or plunger 409).

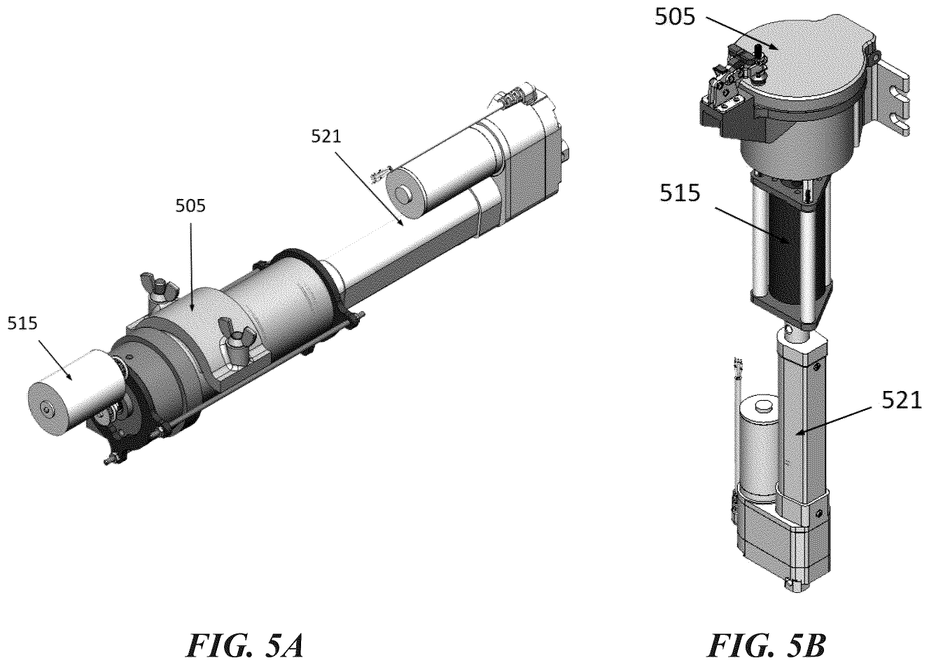

FIGS. 5A-5B schematically illustrate computer aided design (CAD) models of the vessels, coupled to actuators in a horizontal configuration (FIG. 5A) and a vertical configuration (FIG. 5B). The vessels, as described herein, may comprise a motor 515, a door 505, and an actuator 521, such as a linear actuator.

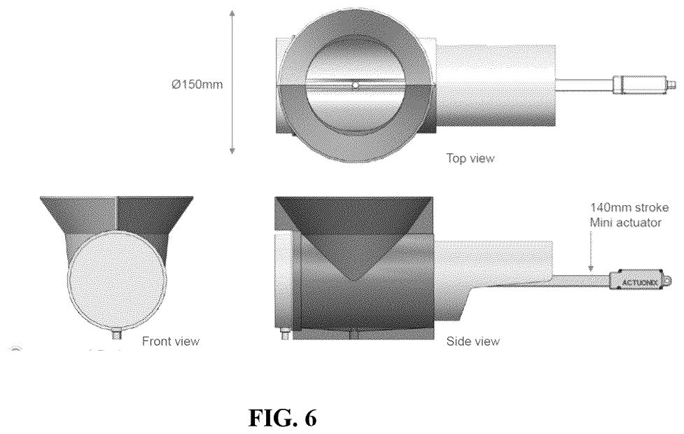

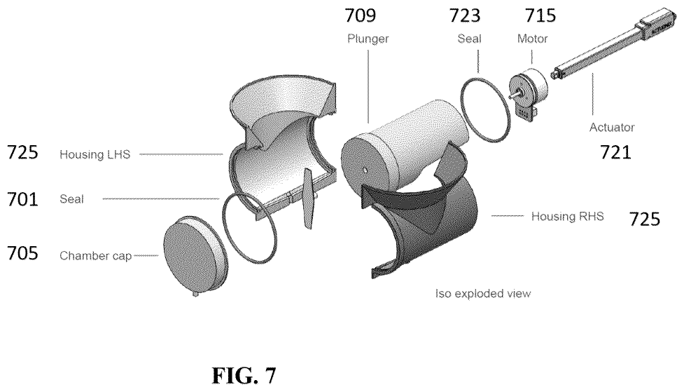

FIG. 6 shows different perspective views (front (i), top (ii), and side (ii)) of another example vessel in a horizontal configuration, and FIG. 7 shows an exploded isometric view of the example vessel of FIG. 6. As described herein, referring to FIG. 7, the vessel may comprise a chamber, which may be surrounded by a housing 725 (shown in left-handed and right-handed sub components), and may further comprise or be coupled to one or more seals 701 and 723, a chamber cap (or door) 705, a plunger 709, a motor 715, a movement actuator 721, etc.

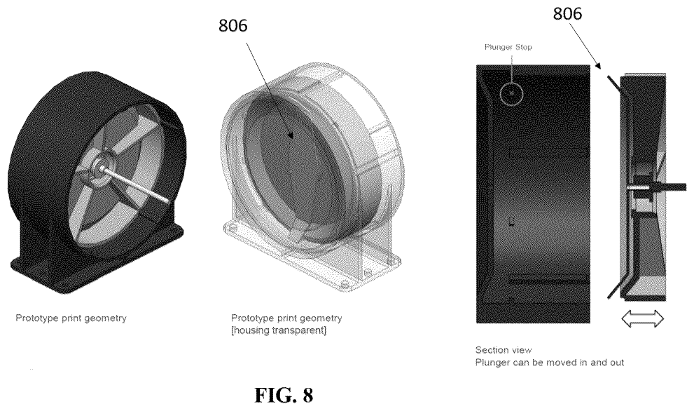

FIG. 8 schematically illustrates a view of the component of the vessel comprising a blade. The blade 806 may be configured to move along the chamber. Features such as grips, stops, or other geometric features may be included to prevent over-extension of the blade 806. The blade 806 may be used to generate the blend, homogenize, agitate, mix, or otherwise move the stool sample, substrate, and liquid to generate a mixture comprising the stool sample, portions of the substrate, and the liquid.

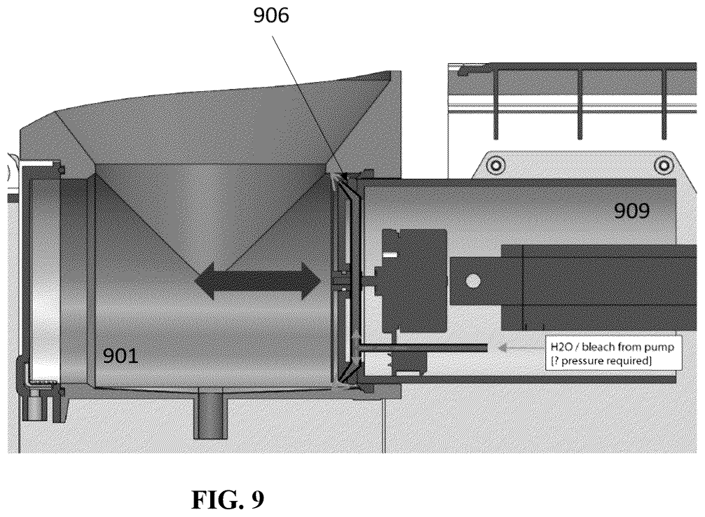

FIG. 9 shows another view of an example vessel in a horizontal configuration. The vessel comprises a chamber 901 and a plunger 909 that is configured to extend toward the chamber 901. The plunger may comprise or be coupled to a blade 906. In some instances, the plunger 909 comprises fluid inlet or outlet ports, which may be useful in transporting fluids (e.g., water, buffers, sanitizing agents) to the chamber 901, e.g., for substrate processing or chamber sterilization. As demonstrated in FIG. 9, the plunger may move relative to the vessel and move the contents contained within the vessel. In some examples, the plunger may move the mixture toward an end of the chamber 901 (e.g., toward an exit port), to expel the contents out of the vessel, e.g., toward another system component for further processing (e.g., lysis of a cell within the mixture, biomolecule extraction, etc.).

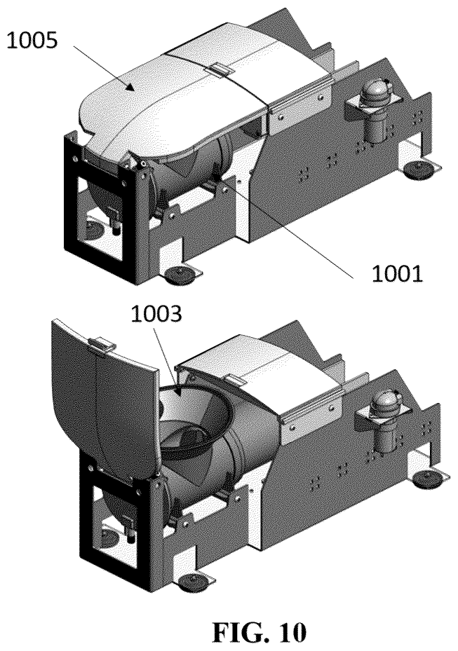

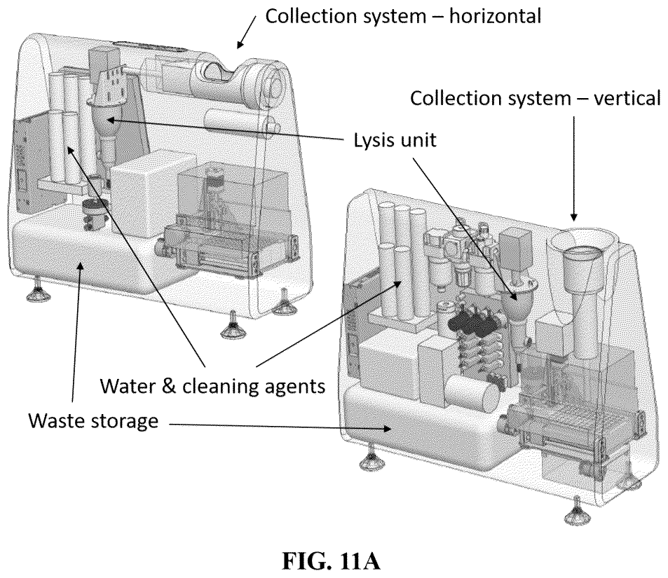

FIG. 10 schematically illustrates a portion of the system comprising the vessel, which may comprise or be coupled to a door 1005 (shown with a latching mechanism). The vessel may comprise a chamber 1001 for processing the substrate comprising the stool sample. In some instances, the chamber 1001 comprises or is coupled to a funnel 1003, which may facilitate entry of the substrate and/or stool sample into the chamber 1001.

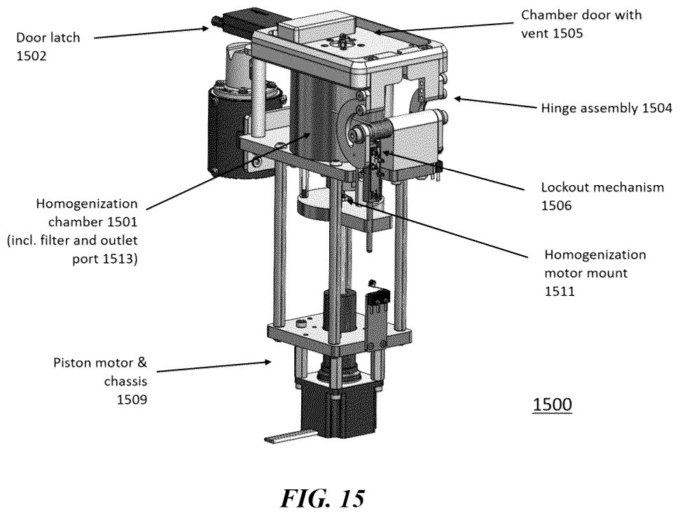

FIG. 15 schematically shows another example of a vessel 1500 of a system described herein. The vessel 1500 may be configured to receive and process a substrate comprising a stool sample. The vessel 1500 may comprise a chamber 1501 in which the substrate and stool sample are received and processed. The vessel 1500 may comprise a lid or door 1505, optionally comprising a vent, which may toggle between an “open” configuration in which the chamber 1501 is accessible to the subject or user and a “closed” configuration, in which the chamber is closed or sealed. The lid or door 1505 may be configured to be opened and closed using a door latch 1502. In some instances, the door latch 1502 may be automatically opened. Opening and closing of the chamber 1501 may, in some examples, be facilitated by a hinge assembly 1504. The vessel 1500 may also comprise a lock mechanism 1506, which may prevent opening of the chamber 1501 during sample processing, e.g., as a safety feature. The lock mechanism 1506 may, in some instances, automatically activate at any convenient time, e.g., at a programmed time, subsequent to depositing of a sample or substrate, subsequent to closing of the lid or door, etc. In some instances, the lock mechanism 1506 may prevent movement of the homogenization motor, e.g., when the lid or door is open.

In some instances, the substrate is processed in the chamber 1501, and the vessel may comprise mechanical parts to facilitate such processing. For example, the vessel 1500 may comprise a piston 1509 which is configured to move the contents of the chamber 1501. The piston 1509 may be configured to move with respect to the chamber 1501 using a variety of mechanisms, such as a screw mechanism, slide bearing, and movement actuator (e.g., motor and chassis). Within or adjacent to the piston 1509, a motor may be provided. Alternatively, or in addition to, a motor may be provided separately from the plunger 1509. For example, the vessel 1500 may comprise a motor mount 1511 configured to couple to an external motor. The motor may be used to drive movement of one or more sample homogenizers, e.g., a blade or propeller, which can be used to mix, homogenize, or agitate the substrate within the chamber 1501. The vessel may comprise fluid inlets that are connected to one or more liquid sources (e.g., water, buffers, sanitizing agents such as bleach or chlorine). The vessel 1500 can comprise additional elements that may be useful for sample processing, such as seals (e.g., gaskets, o-rings, rubber seals), filters, membranes, actuators, fluid inlets and outlets, etc.

The motor and sample homogenizer components (e.g., blades or propellers) may be used to mix, agitate, homogenize, or otherwise mechanically process the substrate comprising the stool sample and the liquid (e.g., water, buffers, etc.), thereby generating a mixture comprising portions of the substrate (e.g., fibers, broken-up pieces of toilet paper, etc.) and the liquid. The mixture may then be directed elsewhere within or out of the chamber 1501, e.g., using the piston 1509. For example, the piston 1509 may move the substrate, stool sample, or mixture thereof to another portion of the system, e.g., a lysis unit, for further sample processing. In one non-limiting example, the piston 1509 may move the mixture against an opposing wall, thereby expunging a liquid comprising a portion of the stool sample (e.g., stool-saturated liquid), which may exit out of the chamber 1501 via a port 1513. In some instances, the port 1513 comprises a filter, which may be used to filter out pieces or portions of the substrate from the mixture, thereby releasing the liquid (e.g., stool-saturated liquid) out of the port 1513. The liquid may then be directed to another portion of the system or output for further processing (e.g., in a laboratory). The vessel 1500 may additionally comprise a port for transporting agents such as waste out of the chamber 1501, e.g., to a different location of the system (e.g., waste unit), or out of the system to a toilet or sewage drain. The vessel 1500 may subsequently be cleaned or disinfected, e.g., using a sterilization agents, e.g., bleach, chlorine, antimicrobial agents, peroxide, ammonia, alcohol, an acid, a base, minerals, soap, etc. or a combination thereof. The sterilization agents may also exit the vessel 1500 via the waste port.

System Activation: The system or portion thereof (e.g., vessel) may be activated automatically or manually (e.g., by a user). For example, the system may comprise one or more sensors which may be used to detect the presence of a subject within a proximity of the device. For example, the sensor may comprise an optical or acoustic sensor and may detect an input, e.g., a change in light or sound, which input may be used to activate or turn on the system. In some instances, the system may be configured to automatically activate. In such cases, the system may be configured to alert the subject (e.g., user) to collect a sample, e.g., by illuminating a light, making a noise, etc. In some instances, the system may comprise a communication interface (e.g., WIFI or Bluetooth communication interface), which may be used to communicate with an electronic device (e.g., phone, computer, tablet, mobile device, etc.). In such instances, the user may be able to activate the device via the communication interface or be alerted (e.g., a notification that sample collection is due).

The subject (e.g., user) may activate the system using one or more mechanisms. For instance, the system may comprise mechanical mechanisms that the user can use to activate the system. Such mechanisms may include, in non-limiting examples, locks, levers, buttons, keypads, springs, motors, switches, pulleys, belts etc. In instances where the system comprises a communication interface, the subject may activate the system using an electronic device. The system may comprise a user interface allowing for activation of the system.

The system may be securely activated, e.g., such that the system activates for specific subjects that meet a security protocol. Such a security protocol may include a biometric protocol. For example, the system may comprise a fingerprint sensor, palm reader, motion or gesture sensor, voice recognition, facial recognition, mobile-communication interface, radiofrequency identification (RFID) system, etc., such that a given individual that meets the security protocol can activate the device. In some instances, the system may comprise a keypad and/or may require a pass code, mobile authentication, specific RFID signature, etc. in order to be activated. Following authentication, the system may be activated or unlocked. For example, the vessel door or lid may be lifted or moved to allow access to the collection chamber. When a user fails authentication, the system may remain deactivated or locked. The system may default to a locked configuration or automatically lock following an event (e.g., depositing of a substrate or sample into the vessel).

Vessel: The vessel may comprise a chamber and may be of any useful geometry. As described herein, in some instances, the vessel is a part of a system that is configured to process the mixture or the processed stool sample. The vessel may be configured to receive a range of substrate amount (e.g., a volume, mass) and types (e.g., toilet paper, tissue paper, wipes, paper products, degradable substrates), any of which may have varying mass, volume, ply, absorbance, texture, type, size, etc. The vessel may be configured to receive a stool sample without a substrate. The vessel may comprise a tube, flask, plate, well array, or other container.

In some instances, the vessel or portion of the vessel (e.g., chamber) comprises a door that may have an “open” (or “unlocked) and “closed” (or “locked”) configuration. The door may be configured to open or unlock manually or automatically. In some instances, the toggling between the two configurations comprises moving the door to a different position. The door can be moved using a variety of mechanisms, e.g., springs, motors, gears, etc. The door may have any useful geometry and dimensions. For example, the door may take any shape such as circular, oval, square, rectangular, rhomboid, triangular, pentagonal, hexagonal, heptagonal, octagonal, etc. In some instances, the door may be configured to provide an opening (e.g., to the chamber) of at least 1 inch, at least 2 inches, at least 5 inches, at least 10 inches, or more. In some instances, the door may be configured to provide an opening of about 1 inch, about 2 inches, about 5 inches, about 10 inches, or more. In some instances, the door may be configured to provide an opening of at most about 10 inches, at most about 5 inches, at most about 2 inches, at most about 1 inch, or smaller. In some instances, a dimension of the chamber (e.g., width, depth, length, diameter, etc.) can be configured to accommodate any useful volume. For example, the depth of the chamber may be at least 1 inch, at least 2 inches, at least 5 inches, at least 10 inches, or more. The depth of the chamber may be about 1 inch, about 2 inches, about 5 inches, about 10 inches or greater. The depth of the chamber may be at most about 10 inches, at most about 5 inches, at most about 2 inches, at most about 1 inch, or smaller.

Sensors: The system or component thereof (e.g., vessel) may comprise one or more sensors. The one or more sensors may detect presence of a sample (or substrate comprising the stool sample) collected in the vessel. For example, the system may comprise a weight or mass sensor, optical sensor, motor torque sensor, etc. The one or more sensors may provide one or more outputs to a processor of the system, which can then initiate another process, e.g., filling the vessel with a processing liquid (e.g., water), and processing the sample (e.g., agitating, mixing, extracting, etc.). The one or more sensors may be configured to detect a failure within the device, e.g., absence of a sample or substrate, blockage, etc. A detected failure may then abort the processing operations or other operations performed by the system and, in some instances, automatically trigger a process, such as a mixing and/or sterilization process. As described elsewhere herein, the one or more sensors may be configured to detect the present of a user and may be used for automatically activating the system or vessel.

Substrate Processing: Subsequent to a subject (e.g., user) depositing a substrate (e.g., toilet paper) comprising a stool sample into the system (e.g., in the vessel or chamber), the system may process the sample, which, in some instances, may be performed automatically. In some instances, the system may sense (e.g., via the one or more sensors) the amount of substrate deposited. The system may sense a mass, volume, density, or optical property (e.g., absorbance) of the substrate and dispense a metered amount of liquid (e.g., water) into the vessel according to the sensed amount of substrate.

The substrate comprising the stool sample may be processed with the liquid. As described herein, such processing may include mixing, agitating, homogenizing, blending, chopping, shearing, applying mechanical (e.g., pressure, hydraulic) force to the substrate and the liquid. In some instances, the mixing or agitating is performed using a blade or propeller. The blade may comprise any useful geometry and type. For example, the blade may be flat, angled, curved, serrated, or comprise any useful topographical features. The blade may be dull or sharp. The system may comprise a single blade or multiple blades. The blade (or plurality of blades) may be coupled to or a part of a movement device (e.g., a plunger). The movement device (e.g., plunger) may move separately or in tandem with the blade and may comprise or be coupled to a movement actuator. The movement device may modulate the volume within the chamber; for example, referring to FIG. 2, the movement of the plunger toward the opposing wall decreases the volume of the chamber. The movement of the plunger may facilitate homogenization, mixing, or agitation of the contents within the chamber, which may be useful in limiting motor torque and reducing obstructions or jamming. The processing of the substrate and the liquid may result in a mixture comprising the liquid, portions (e.g., fibers, pieces) of the substrate, and the stool sample.

The movement device (e.g., plunger) may further process the mixture. For example, the movement device may be used to compress the mixture (e.g., by directing the movement device within the chamber, toward an opposing wall), thereby separating or releasing at least a portion of the stool sample from the mixture. By way of example, the movement device may be a plunger which moves the mixture toward an opposing wall and decreasing the chamber volume. The mixture may be compressed against the opposing wall, thereby expelling or extracting stool-saturated liquid from the substrate portions (e.g., tissue paper fiber or pulp). The stool-saturated liquid, which comprises at least a portion of the stool sample, may then be transported out of the chamber. The transportation of the stool-saturated liquid may be achieved through any useful mechanism, e.g., vacuum or application of pressure, the use of a pump (e.g., peristaltic pump, diaphragm pump), an air compressor, etc. In some instances, the stool-saturated liquid is transferred to another processing unit of the system, such as a processing unit to conduct cell lysis, sample storage and/or preservation or stabilization, or output from the system for further processing, e.g., by a clinician or laboratory technician. Additional examples of systems and methods for processing stool samples, sample storage, and cell lysis may include the systems and methods disclosed in International Patent Application No. PCT/US20/38563, filed Jun. 18, 2020, which is incorporated by reference herein in its entirety.

Cleaning: Subsequent to the extraction of the stool-saturated liquid comprising at least a portion of the stool sample, the vessel may be subjected to conditions sufficient to clean or sterilize the vessel. Such conditions may comprise any useful cleaning or sterilization process, such as heating, optionally with high pressure (e.g., autoclaving), flushing of the vessel (or chamber) with cleaning or sterilization agents, irradiation (e.g., UV, gamma, or X-ray irradiation). Examples of cleaning or sterilization agents include chlorine, bleach, hydrogen peroxide, soaps, surfactants, detergents, ammonia, alcohol, acid, base, etc. In some instances, the cleaning or sterilization agents may be stored in or adjacent to the system and diluted to a relevant concentration in the vessel during the cleaning or sterilization. In such examples of chemical sterilization, the movement device (e.g., plunger) may be restored to its original position (i.e., opposite the opposing wall), and the blades or propellers may be used to mix or circulate the sterilization agent within the chamber. In some instances, the movement device may be configured to move within the chamber (e.g., back to the original position, or back-and-forth within the chamber), while the cleaning or sterilization agents are added. Mixing or blending conducted by the blades may be used to efficiently circulate the cleaning or sterilization agents within the chamber. Waste products may be expelled through a waste port, optionally with additional water rinses. The expelling of the waste may be performed using gravitational force or other applied force (e.g., air pressure, water pressure, etc.). In some instances, the expelled waste may be temporarily stored within a storage area of the system (e.g., a removable container), which the subject may separately remove. In some instances, the expelled waste may be directed to the waste or sewage drainage from a toilet fluid line, or the expelled waste may be directed to a toilet. The cleaning processes, or certain operations of the cleaning process may be performed manually or automatically. In some instances, one or more sensors may be used to determine when cleaning or sterilization is complete (e.g., via optical or weight sensors to determine the amount of substrate or stool sample remaining in the chamber or waste outlets).

Volumes: Any useful volume of liquid used in the processes described herein may be used. For example, for mixing, agitation, or homogenization of the substrate comprising the stool sample and the added liquid (e.g., water or buffers), the amount of liquid added may be about 1 milliliter (mL), about 5 mL, about 10 mL, about 50 mL, about 100 mL, about 200 mL, about 500 mL or more. The amount of liquid may fall within a range of volumes, e.g., from about 500 mL-1 L, or from about 50 mL-500 mL. During cleaning, the amount of water used may be any useful amount; for example, the amount of water that may be used may be about 10 mL, about 50 mL, about 100 mL, about 500 mL, about 1 L, about 2 L, about 3 L, or more. The volume of agents (e.g., water, sterilization agents) used for cleaning may be adjusted to achieve a useful concentration, e.g., 100-5000 ppm bleach, 50-70% alcohol, etc. The volumes used within the systems and methods described herein may be measured or controlled, e.g., using fluid control or handling units, metered volume units, etc., as described elsewhere herein.

Processing Duration: Any of the processes described herein may be performed for any useful duration of time. For example, the mixing or agitation of the substrate with the liquid may be about 10 seconds (s), about 20 s, about 30 s, about 40 s, about 50 s, about 1 minute (min), about 5 min, about 10 min, about 20 min, about 30 min, or more. The cleaning or sterilization may occur for about 30 s, about 1 min, about 5 min, about 10 min, about 20 min, about 30 min, or more.

Substrate: The substrate may be any useful material for collecting or processing a stool sample. In some instances, the substrate comprises toilet paper. The substrate may be a porous material. The substrate may be an absorbent material. The substrate may have any useful material properties, such as a designated porosity, absorbance, dissolvability in a liquid (e.g., water, buffer), solubility under a defined condition (e.g., pH, salt content, temperature, etc.). The substrate may comprise a paper product (e.g., paper towel, wipe, napkin, tissue paper, toilet paper, etc.). The substrate may comprise a polymer, which can be naturally occurring or synthetic.

System Components: The system may be configured to perform multiple functions for processing the substrate comprising the stool sample. For example, the system may be configured to extract a biomolecule from the stool sample, using the processes described herein. In such examples, the system may comprise additional units for further processing of the substrate, the stool sample, the processed substrate (e.g., a homogenized mixture of the substrate, a liquid, and the stool sample), or derivatives thereof. For example, the systems and methods described herein may include the systems and methods described in International Patent Application No. PCT/US20/38563, filed Jun. 18, 2020, which is incorporated by reference herein in its entirety.

By way of example, the system may comprise units for processing the stool-saturated liquid (e.g., following mixing or agitating of the substrate with a liquid and extracting a portion of the stool sample via compression of the mixture to expel the stool-saturated liquid). The stool sample may be filtered and transported to a lysis unit (e.g., lysis chamber) contained within or separate from the system. A portion of the stool sample (e.g., in the stool-saturated liquid) or derivative thereof may be further processed, e.g., via lysis of any cells (e.g., bacterial, fungal, viral, human, animal) present in the stool sample. The lysis may comprise mechanical lysis (e.g., using a motor and an impeller, beads, and/or other mechanical lysis mechanisms) or chemical lysis (e.g., via the addition of detergents such as sodium dodecyl sulfate, Triton-X 100, etc. or other lysis agents).

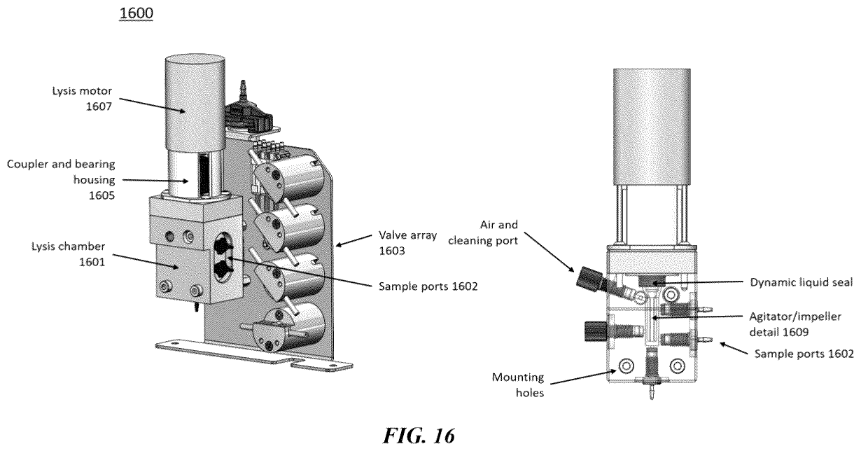

FIG. 16 schematically illustrates an example of a lysis unit described herein. The lysis unit may be configured to receive the stool sample (or stool-saturated liquid), lyse the cells contained therein to generate a lysed product, meter a volume of the lysed product to another unit (e.g., storage unit) of the system, or a combination thereof. The lysis unit 1600 may comprise a lysis chamber 1601 which comprises one or more ports, e.g., for allowing entrance or exit of the sample (e.g., the stool sample or stool-saturated liquid) into or out of the lysis chamber 1601. The lysis chamber 1601 may be adjacent or in communication with a valve array 1603, which may control entrance or exit volume of the sample, volume of the processing reagents (e.g., buffers), etc. The lysis chamber 1601 may be coupled to a housing 1605, which may comprise a coupler and/or bearing to couple to the lysis chamber 1601 and, in some instances, a motor 1607. The lysis chamber may comprise or be configured to couple to an agitator or impeller 1609. For example, the lysis chamber may comprise an agitator or impeller 1609, which may interact with the lysis motor 1607, e.g., to rotate or move the agitator or impeller 1609 to facilitate lysis. In some instances, the lysis chamber comprises or may be configured to use one or more supports to perform the lysis. For example, beads may be provided to the lysis chamber, and the impeller coupled to the motor may move the beads within the lysis chamber to perform lysis of cells within the chamber. The lysis chamber 1601 may comprise any other useful features, e.g., a seal (e.g., dynamic liquid seal, gasket, O-ring, etc.), mounting structures (e.g., holes, hooks, interlocking parts, press fit components, threads, screws, etc.), one or more ports (e.g., for introducing liquids such as buffers, lysis reagents, sanitization agents, air, etc.). Following lysis, a metered volume of the lysed product may be provided to another unit of the system, e.g., a storage unit or isolation (or extraction) unit.

In some instances, the system comprises an isolation unit used for extracting a biomolecule from the substrate or the stool sample. The isolation unit may be in fluidic communication with the lysis unit or may be performed in the lysis unit. Alternatively, the isolation unit may be separate from the lysis unit. In some instances, the isolation unity may be a part of a storage unit, such that isolation and storage may occur in the same location. Such an isolation unit may comprise a binding buffer and beads (e.g., comprising capture molecules, such as nucleic acid molecules, proteins, antibodies, etc. that are configured to bind to a biomolecule) or other capturing techniques, e.g., chromatography, filters, columns, etc. For example, the stool sample or derivative thereof (e.g., stool-saturated liquid) may be mixed with the beads and binding buffer. The beads may be used to capture a biomolecule within the sample, such as cells, organelles, nucleic acid molecules (e.g., DNA or RNA), proteins, peptides, lipids, carbohydrates, metabolites, or a combination thereof.

In some instances, the system comprises a storage unit. The substrate or derivative thereof (e.g., substrate portions, mixture of substrate with liquid, homogenized substrate-liquid mixture, a portion of the stool sample, stool-saturated liquid, lysed product, etc.) may be transported to a storage unit. Alternatively or in addition to, the storage unit may be configured to contain the isolated or extracted biomolecules (e.g., biomolecules in solution, bead-bound biomolecules). Further processing may be performed in the system, either in the lysis unit, the isolation or extraction unit, or the storage unit, or a combination thereof. For example, the further processing may include rinsing of beads with wash buffer. The wash buffer may be removed and the beads may optionally be dried in process. In some instances, when beads are used, an elution buffer may be transported to the isolation unit or the storage unit. The elution buffer may be mixed with the beads and the eluted (or otherwise extracted or isolated) biomolecule may be transported to the storage unit or a different unit. The storage unit may comprise any preservatives or storing agents, such as stabilization buffers.

In some instances, the lysed product from the lysis unit may be directed to a storage unit for further processing (e.g., extraction or isolation of a biomolecule). For instance, the system may comprise a fluid handling system that is configured to direct the lysed product into a sample container in the storage unit for further processing. The fluid handling system or the storage unit may comprise a moveable stage (e.g., a motorized stage, an automated stage, etc.). The stage may be configured to move in any direction (e.g., X-Y-Z axes). The fluid handling system may be in fluidic communication with agents used for isolation or extraction of a biomolecule, including, in non-limiting examples, binding buffers, wash buffers, elution buffers, etc.

The sample container may take any useful geometry and may comprise a tube, vial, well, plate, flask, etc. The sample container may contain an array of containers, for instance, a multi-well unit, an array of tubes or flasks, a microchip array configured to capture beads, etc. or an array for analyzing the biomolecule (e.g., microarray). In some instances, the collection unit comprises a multi-well unit, and each well is configured to hold a sample taken at a time point; accordingly, the multi-well unit may be amenable for performing longitudinal sample collection, in which multiple samples are collected over a period of time and distributed, at each time point, into a single well or a plurality of pre-defined wells.

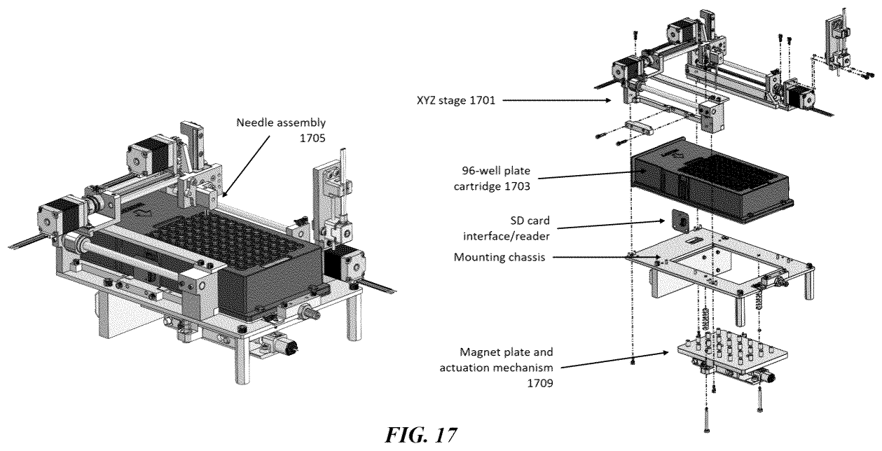

FIG. 17 schematically shows an example of components of a storage unit of the systems described herein. The storage unit may comprise a moveable stage 1701, which may be configured to move in any direction (e.g., X-Y-Z axes) and may be configured to couple to a sample container 1703, e.g., a multi-well plate such as a 96-well plate. In some instances, the moveable stage 1701 may comprise or be coupled to a fluid handling unit 1705. The fluid handling unit may be configured to aliquot or direct the lysed product, or portion thereof, to the sample container and optionally, perform further processing. For example, the fluid handling unit 1705 may comprise a needle assembly, which may be useful in providing reagents to the multi-wells (e.g., to perform extraction or isolation of a biomolecule) and/or mixing or agitating of the contents in the multi-wells. The storage unit may further comprise a mount or other positioning mechanisms (e.g., to position the sample container). In some instances, the storage unit may comprise or be configured to couple to a memory device, e.g., a secure digital (SD) memory card. Such a memory device may be used to store information about the sample (e.g., date, time collected, volumes processed, etc.).

In some instances, the sample container may comprise or be coupled to reagents for performing isolation or extraction of a biomolecule. For example, the sample container may be in fluidic communication (e.g., via the fluid handling unit) with reservoirs comprising reagents for DNA, RNA, or protein isolation. Such reagents may comprise, for example, binding reagents, wash buffers, elution buffers, capture particles (e.g., beads), etc. In some instances, the fluid handling unit comprises a needle assembly, which may be useful for distributing reagents to the sample container and agitating or mixing the contents within the sample container. In some instances, magnetic beads may be used for isolating or extracting one or more biomolecules from the lysed product; as such, the storage unit may optionally comprise a magnetic device and actuator (e.g., magnetic surface and actuation mechanism).

In one non-limiting example, the sample container may be used for performing isolation or extraction of a biomolecule and also storing the isolated or extracted biomolecule. In such an example, the lysed product from the lysis unit may be directed to the sample container (e.g., a multi-well plate) via the fluid-handling unit 1705. Reagents for biomolecule isolation or extraction may be provided to the sample container, e.g., biomolecule-capturing beads (e.g., antibody-coated beads, oligo-capturing beads, etc.), along with necessary reagents, such as binding buffers, wash buffers, elution buffers, etc. In some instances, the storage unit (e.g., fluid handling unit comprising a needle assembly) may comprise aspirating units, e.g., to remove waste products from the sample containers, to facilitate extraction or isolation of a biomolecule in the sample container.

The sample container (e.g., multi-well plate) may comprise biomolecule probes (e.g., antibodies, aptamers, nucleic acid probes, etc.). Following isolation of the biomolecules in the sample container, the probes may be used to indicate the presence, identity, quantity or other metric of the biomolecules in the stool sample. For instance, nucleic acid or protein probes may be immobilized (covalently or non-covalently) to the sample container (e.g., a surface of a well). The isolated or extracted biomolecule may bind or couple to the nucleic acid or protein probes and form a detectable product (e.g., a precipitate, a fluorescent product, a chemiluminescent or other luminescent product, etc.), optionally with the addition of other reagents (e.g., additional antibodies, fluorescent or colorimetric probes, etc.). For example, the sample container may comprise reagents or have access to reagents for performing an assay, such as fluorescence in situ hybridization (FISH) or an enzyme-linked immunosorbent assay (ELISA) or sandwich ELISA for detection of at least one biomolecule. Multiplexed assays (e.g., for measuring or analyzing different types of biomolecules such as DNA and protein) may be performed in the sample containers. In some instances, further processing may be performed in the sample containers (internally or externally of the collection unit). Non-limiting examples of processing operations include, for instance: filtration, heating, cooling, applying an electric potential, separation of one or more analytes (e.g., via filtration, chromatography, electrokinetics, centrifugation), partitioning (e.g., forming droplets), flow cytometry, merging of reagents, mixing, interfacing with an analysis instrument (e.g., mass spectrometer, sequencing instrument, etc.). The processing operations may be performed in any combination or order.

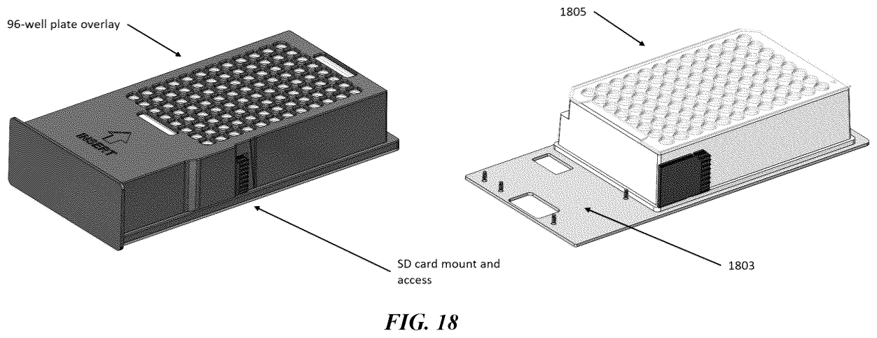

FIG. 18 schematically shows an example of a sample container for use in the systems and methods described herein. The sample container may comprise or be configured to couple to a multi-well plate. The sample container may comprise a cartridge, which may comprise a rigid substrate 1803. The rigid substrate may be configured to couple to the multi-well plate 1805, which in some instances, may be a disposable or replaceable element. The sample container may any other useful features, e.g., a memory component (e.g., an SD card slot), and any other features that may be beneficial in sample preparation, e.g., an overlay, a septum, seals, gaskets, etc., which may be useful in preventing cross-contamination of samples. In some examples, the sample container may comprise a pierceable membrane or septum (e.g., silicone septum), which may separate a sample (or extracted biomolecule) from the surrounding environment.

As described herein, any portion of the system may be cleaned, e.g., using water, bleach, sterilization agents etc. For example, the vessel comprising the collection chamber, the lysis unit and/or any components contained therein (e.g., beads, impeller, lysis chamber, etc.) may be sterilized. In such examples, the system may comprise one or more reservoirs or storage units for storing the sterilization or cleaning agents.

Waste from the system, e.g., from the vessel (e.g., excess substrate-liquid mixture), the lysis unit, or the storage unit, may be deposited in a waste unit, or in the chamber and expelled into the toilet drain. In some instances, the system comprises a waste unit which is configured to temporarily store any waste products (e.g., portions of the substrate, liquid waste) from any of the units. The waste unit may be coupled to a waste receptacle or toilet (e.g., toilet bowl) and may facilitate ejection of waste products. The waste unit may comprise a mechanism for moving the contents contained therein, e.g., pneumatic agitators or pumps, pressurized water or gas, or automated moving parts (e.g., pulleys, gears, screws, motors, vacuums) for moving the waste unit to the waste receptacle or toilet. The waste unit may comprise one or more access ports or fluidic inlets and outlets, which may optionally be coupled to other agents, e.g., sterilization agents, scented agents, color-modulating agents, neutralizing agents. In some examples, the waste unit may be coupled to a reservoir comprising such agents. As described herein, any combination or all of the processes described herein may be performed automatically.

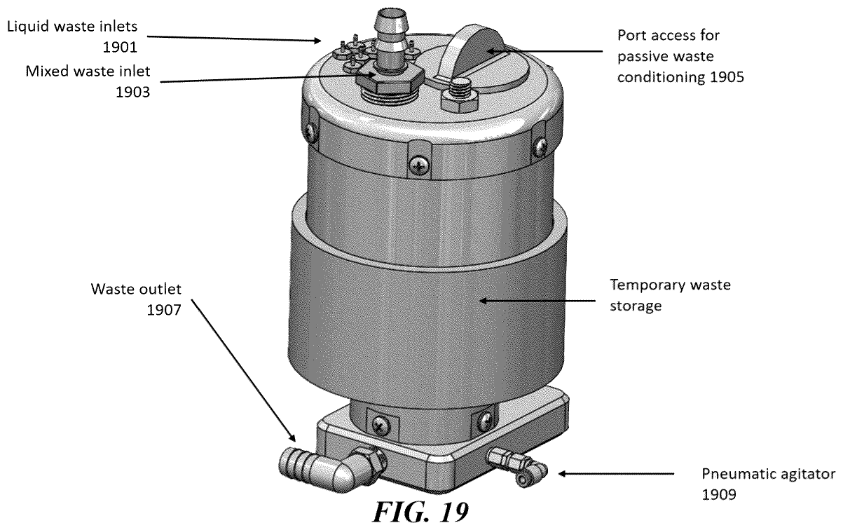

FIG. 19 shows an example of a waste unit. The waste unit may take any suitable geometry, e.g., cylindrical, rectangular, spherical, rhomboidal, triangular, prism, etc. The waste unit may be configured to house or store temporary waste. Waste products, e.g., from the lysis unit or the vessel, may be provided to the waste unit via one or more waste inlets 1901 and 1903. In some examples, the waste unit comprises one or more types of inlets, e.g., a liquid waste inlet 1901 and a waste inlet 1903, e.g., for containing solid particles (e.g., portions of substrate). The waste unit may comprise one or more ports 1905, which may allow for entry of other agents (e.g., sterilization agents, scented agents, etc.). The waste unit may comprise a waste outlet 1907, which may be coupled to a waste receptacle, toilet (e.g., to a toilet bowl), or sewage line. In some instances, the waste unit may comprise a mechanism for moving or evacuating the waste unit, e.g., a pneumatic agitator 1909.

The system may include one or more motors. The motors may be any suitable type of motor, e.g., an electric motor, a manual motor, an air motor, etc. In some instances, the motors may be coupled to one or more gears and/or one or more pumps. One or more motors may be used for a variety of purposes, including but not limited to: opening and/or closing or the vessel or chamber, extension and/or retraction of the movement unit (e.g., plunger), homogenization, mixing, or agitation of the substrate comprising the stool sample, optionally with a liquid, pumping a fluid or reagent into the chamber, directing the stool sample or processed stool sample (e.g., substrate pieces or portions and stool sample) away from the chamber and to a different unit within the system.

As described herein, the vessel may be coupled to one or more fluid inlets or fluid flow paths. One or more reagents (e.g., water, buffers, sample processing solutions, etc.) may be introduced into the chamber (e.g., via the fluid inlet). The chamber may be used to chop, blend, or shred the substrate and generate a mixture comprising the liquid and the portions of the substrate. The blades may be coupled to a motor. The substrate comprising the stool sample (or just a stool sample) may be deposited into the chamber, and a homogenization buffer may be introduced (e.g., filtered water). The stool sample and the substrate may be homogenized by actuation of the motor coupled to one or more blades to stir, blend, rotate, spin, move, and/or homogenize the sample. In another example, the chamber may comprise one or more pumps (e.g., a centrifugal pump, displacement pump, rotary pump, piston pump, rotary lobe pump, rotary gear pump, diaphragm pump, screw pump, gear pump, vane pump, peristaltic pump, or a combination thereof) to generate a pressure (e.g., water pressure) to homogenize the substrate-liquid mixture comprising the sample (e.g., via mixing, vortexing, etc.). The chamber may comprise a homogenizer, e.g., a rotor stator homogenizer, mechanical mortar, blender, masher, mills (e.g., bead mills), rollers, mixers, and/or beads (e.g., magnetic beads). One or more forces (e.g., centrifugal, gravitational, positive or negative pressure, ultrasonic, magnetic etc.) may be applied to the substrate, sample, reagents, and/or other homogenizer materials (e.g., applying a magnetic force to magnetic beads) to homogenize the mixture. Alternatively or in addition to, the chamber may comprise a heating or cooling element, and the sample may be heated or cooled to generate the mixture.

The system may be coupled to and/or comprise one or more fluid inlets and outlets. A fluid inlet may be used to introduce one or more reagents to the system (e.g., the vessel, the lysis unit, the storage unit, etc.). For example, the fluid inlet may be used to introduce water, a buffer, or other reagent into the vessel, via a coupled fluid flow path. The fluid inlet of the vessel or portion thereof (e.g., chamber) may be coupled to a fluid flow path, e.g., via a hose, pipe, funnel, etc. The fluid inlet may be coupled to or in fluid communication with a source of reagents (e.g., water, buffers, sterilization solutions, etc.). The fluid inlet may comprise one or more valves (e.g., y-valve, y-tube, T-valve, etc.) that may control the flow volume or rate of the reagents into system. In some instances, the system is coupled to and/or further comprises a fluid outlet, which may be used to direct the stool sample or processed substrate away from the system to be processed, and/or analyzed. The sample outlet may be used to direct the substrate, the processed stool sample (e.g., stool-saturated liquid) to a collection unit, to a sensor for sample analysis, or out of the system for analysis (e.g., in a laboratory). Such analysis may include, in non-limiting examples, PCR or nucleic acid sequencing, mass spectrometry or other proteomic measurement tool, Raman spectroscopy, etc.

In some instances, the fluid flow path may comprise a plurality of valves that may be used to control the flow volume or rate of a fluid into or out of the system, or portion thereof. In some cases, it may be useful to have a valve that can interface with more than one inlet or outlet or portions of the fluid flow path. In such cases, a custom-built valve may be used.

In some instances, the system comprises a metering system, which may be coupled to one or more fluid inlets or outlets. The metering system may comprise, in some examples, a buffer metering manifold. Such a manifold may be useful in directing reagents to and from components of the system (e.g., the vessel, lysis unit, waste unit, storage unit, reservoirs, etc.). The metering system may comprise a pump, a pneumatic interface, an inlet, an outlet, a valve, interfaces for other components of the system, or a combination thereof. Such a metering system may be useful in directing one or more fluids (e.g., buffers, sanitization agents, sample isolation or extraction fluids, etc.) to the appropriate system components (e.g., the vessel, lysis unit, waste unit, storage unit, etc.). In some instances, the metering system measures or meters specific volumes of fluids (e.g., liquids or gases). For example, the metering system may meter specific volumes of buffers or supply specific pneumatic pressures to a system component.

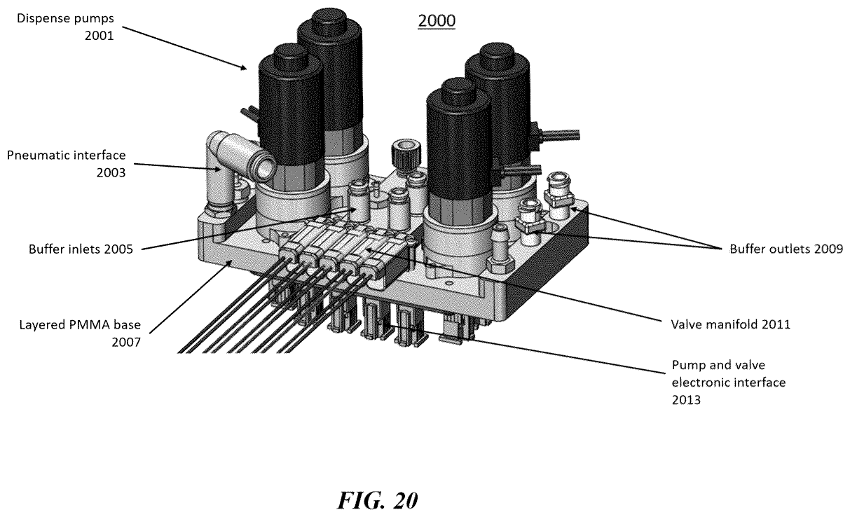

FIG. 20 shows an example of a metering system. The metering system 2000 may comprise one or more pumps 2001, a pneumatic interface 2003, a buffer inlet 2005, a buffer outlet 2009, a valve manifold 2011, a base 2007, a pump and/or valve electronic interface 2013, or any combination thereof. The pumps 2001 may be in fluidic communication with a reservoir, e.g., comprising buffers, sanitization agents, biomolecule isolation or extraction reagents, etc. and may be used to measure or meter specified volumes of such agents to the relevant system component (e.g., sanitization agents to the vessel and/or waste unit, lysis buffers to the lysis unit, etc.). The pneumatic interface may be coupled to a gas line or pump and may be used, in some instances, for directing the waste from the waste unit out of the system (e.g., to a toilet bowl or sewage line). The buffer inlets 2005 and outlets 2009 may be used to direct agents across the system, e.g., from a reservoir to the relevant system component. The metering system 2000 may also comprise electronic interfaces 2013, e.g., configured to interface with a computer processor or other electronic device, which may be used to automate the metering volumes and process.

The system (e.g., metering system) may comprise a pump connected to the fluid flow path. The pump may be a rotary pump, gear pump, piston pump, diaphragm pump, screw pump, vane pump, peristaltic pump, centrifugal pump, etc., or variations or a combination thereof. In some cases, the reagents may be directed to the fluid flow path using an applied or generated force, e.g., hydraulic, centrifugal, gravitational, frictional, tensional, spring, pneumatic, etc. One or more parts may be coupled to a power supply. Alternatively or in addition to, one or more parts may be configured to couple to an electrical outlet.

The system may comprise a communication interface that allows for transmitting and/or receiving data corresponding from a portion of the vessel. In some cases, the data may be transmitted to an electronic device in communication with the communication interface. The communication interface may be a wireless communication interface, e.g., a Wi-Fi interface, a near-field communication interface, or a Bluetooth interface. The electronic device may be a device that may communicate with the communication interface. The electronic device may be a mobile device (e.g., a smart phone, tablet, laptop, etc.). Alternatively or in addition to, the communication interface may be a wired communication interface. The stool collector may comprise a port for communication and/or a power supply (e.g., universal serial bus (USB), USB-type C, Thunderbolt, etc.).

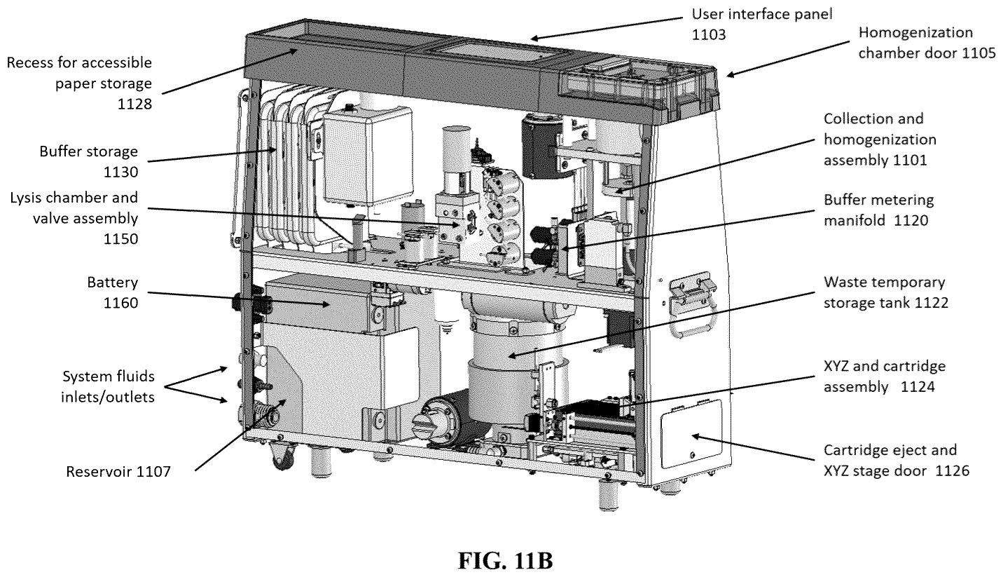

FIG. 11A schematically illustrates an example system described herein. The system may comprise a vessel (referred to as a “collection system”), which may be in a horizontal or vertical configuration. The vessel, as described herein, may be configured to collect a substrate comprising a stool sample (or just a stool sample) and process the substrate (or stool sample) with a liquid, thereby generating a mixture of portions of the substrate with the liquid. The mixture may be compressed in a chamber of the vessel, and the stool-saturated liquid may be directed to another unit of the system (e.g., a lysis unit). The lysis unit may comprise agents for lysing a cell within the stool sample (or stool-saturated liquid), including mechanical or chemical mechanisms for lysis. The lysed product may then be directed to a storage unit within the system. Waste products may also be stored in a waste storage unit within the system or, alternatively expelled. The system may also comprise containers for housing water, liquids, buffers, cleaning agents, or other reagents.

FIG. 11B schematically illustrates another example system described herein. The system may comprise a user interface 1103 (e.g., a panel comprising sensors, or user interaction features). The user interface 1103 may be configured to automatically turn on or off any components within the system. For example, the user interface 1103 may automatically detect a subject and render the collection chamber 1101 accessible, e.g., by opening or unlocking a door 1105 to the vessel comprising a collection chamber 1101. The system may comprise a vessel for sample collection and processing. The vessel may be configured to collect, e.g., in a chamber 1101 (“collection and homogenization assembly”), a substrate comprising a stool sample (or just a stool sample) and process the substrate (or stool sample). Such processing may comprise, for example, providing a liquid (e.g., water, buffer), which optionally may be stored within the system and provided to the vessel 1101 via a reservoir 1170 comprising fluid inlets and/or outlets connected to the collection chamber 1101. Alternatively or in addition to, liquids may be provided to the collection chamber 1101 via other inlets or outlets, e.g., from an external source (e.g., a water source from a nearby sink or toilet). The liquid may be measured and provided in precise volumes using a buffer metering manifold 1120 into the chamber 1101. The liquid and substrate comprising the sample (or just the sample) may be processed, e.g., mixed, agitated, homogenized, etc. within the chamber 1101 to generate a mixture comprising the liquid and portions of the substrate.

The mixture may be further processed. For example, the mixture may be compressed in the chamber 1101, and the stool-saturated liquid may be directed to another unit of the system (e.g., a lysis unit 1150). The lysis unit 1150 may comprise a valve or other fluid control mechanisms. The lysis unit may comprise or be fluidically connected to a reservoir (e.g., the same, different, or additional reservoir as 1107), e.g., a buffer storage reservoir 1130, comprising agents for lysing a cell within the stool sample (or stool-saturated liquid). The lysis unit 1150 may comprise mechanical (e.g., blades, beads, etc.) or chemical mechanisms (e.g., using detergents or chaotropes) for lysis to generate a lysed product. Alternatively or in addition to, the lysis unit may comprise mechanisms to process the stool sample (or stool-saturated liquid) to extract one or more biomolecules (e.g., nucleic acids such as DNA or RNA, proteins, metabolites, etc.). The lysed product may then be directed to a storage unit within the system. For example, the system may comprise a cartridge or other storage vessel, which may be integrated with or provided to the system. In some instances, the system may comprise a mechanical stage (“XYZ and cartridge assembly”) 1124, which can move the cartridge or storage vessel within the system, e.g., along 1, 2 or 3 axes. The cartridge or storage vessel may be separately accessible and/or replenishable via a stage door 1126. In some instances, the cartridge or storage vessel may comprise commercially available products, such as multi-well plates, tubes, etc.

Waste products, e.g., from the collection chamber 1101 or the lysis unit 1150 may also be directed and stored in a waste unit 1122 within the system or, alternatively expelled. In some instances, the waste products may be dispensed into a toilet or be connected to a sewage line. The system may also comprise containers for housing water, liquids, buffers, cleaning agents, or other reagents, e.g., in one or more reservoirs 1107 and 1130. Alternatively or in addition to, the system may comprise one or more inlets or outlets for connecting to external fluid sources, e.g., a water source.

The system may comprise other useful components such as an external power source (e.g., plug for electricity) and/or a battery 1160. The system may also be configured to store or provide the substrate (e.g., toilet paper), e.g., via a substrate storage area 1128.

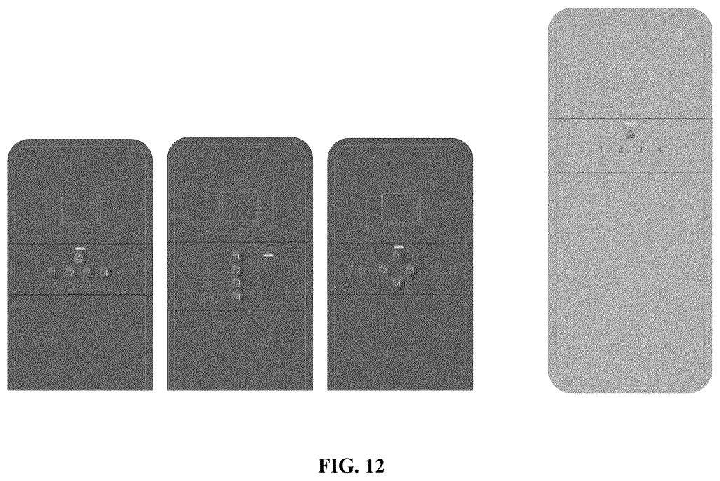

FIG. 12 shows an example of a user interface of the system, which may be coupled to or part of a housing of the system. The user interface may comprise electronics and may comprise a keypad (e.g., for activation of the device). In some instances, the user interface comprises one or more lights or indicators to indicate which stage or processing is occurring or to display any system errors that have occurred.

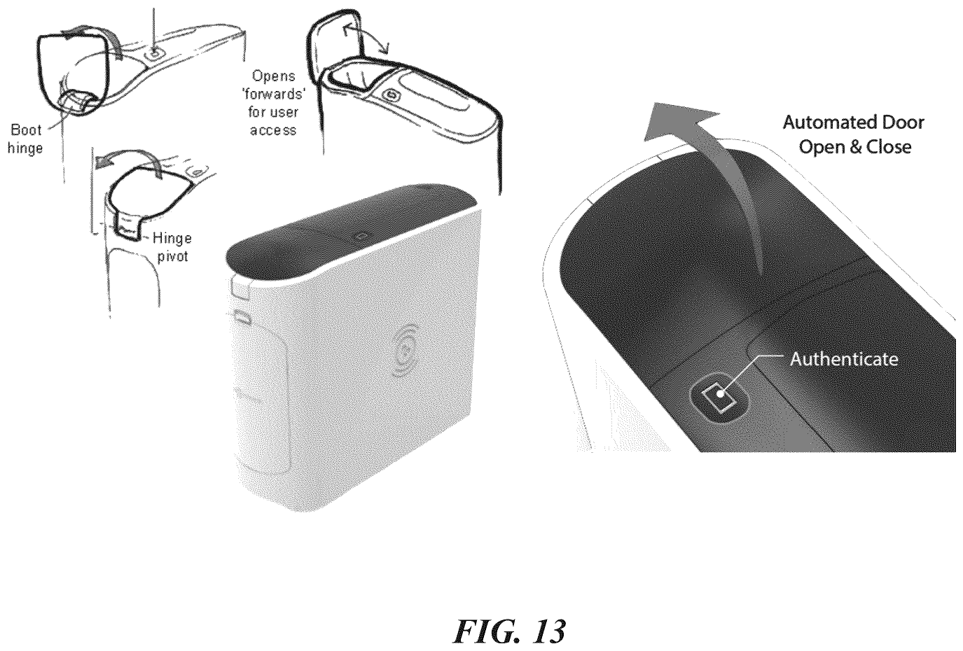

FIG. 13 illustrates an example system as described herein. The system may comprise a housing comprising a door. The door may be toggled to an “open” configuration (e.g., following user authentication), thereby granting access to the collection vessel. The door may be automated (e.g., via a motion sensor, voice control, etc.), or the door may be manually operated.

### Computer Systems

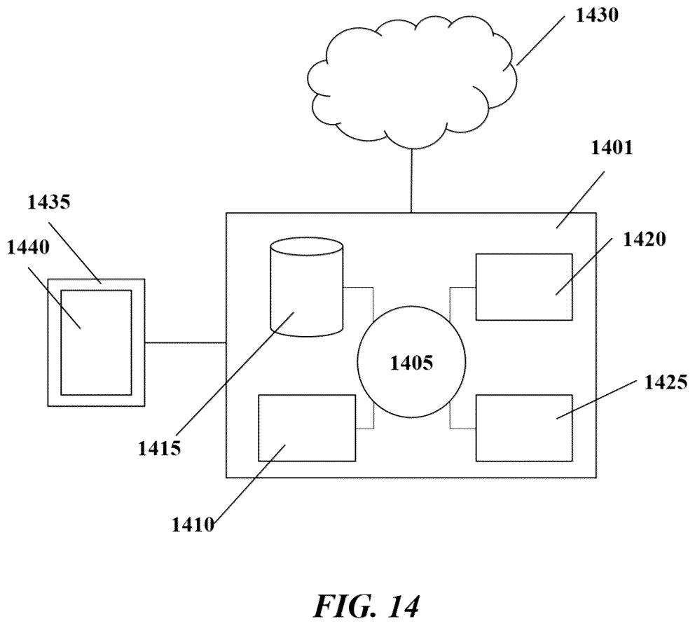

The present disclosure provides computer systems that are programmed to implement methods of the disclosure. FIG. 14 shows a computer system 1401 that is programmed or otherwise configured to automate a process disclosed herein. The computer system 1401 can regulate various aspects of sample collection of the present disclosure, such as, for example, monitoring collection of substrate or stool sample collection, receiving and/or storing an input from one or more sensors, etc. The computer system 1401 can be an electronic device of a user or a computer system that is remotely located with respect to the electronic device. The electronic device can be a mobile electronic device.

The computer system 1401 includes a central processing unit (CPU, also “processor” and “computer processor” herein) 1405, which can be a single core or multi core processor, or a plurality of processors for parallel processing. The computer system 1401 also includes memory or memory location 1410 (e.g., random-access memory, read-only memory, flash memory), electronic storage unit 1415 (e.g., hard disk), communication interface 1420 (e.g., network adapter) for communicating with one or more other systems, and peripheral devices 1425, such as cache, other memory, data storage and/or electronic display adapters. The memory 1410, storage unit 1415, interface 1420 and peripheral devices 1425 are in communication with the CPU 1405 through a communication bus (solid lines), such as a motherboard. The storage unit 1415 can be a data storage unit (or data repository) for storing data. The computer system 1401 can be operatively coupled to a computer network (“network”) 1430 with the aid of the communication interface 1420. The network 1430 can be the Internet, an internet and/or extranet, or an intranet and/or extranet that is in communication with the Internet. The network 1430 in some cases is a telecommunication and/or data network. The network 1430 can include one or more computer servers, which can enable distributed computing, such as cloud computing. The network 1430, in some cases with the aid of the computer system 1401, can implement a peer-to-peer network, which may enable devices coupled to the computer system 1401 to behave as a client or a server.

The CPU 1405 can execute a sequence of machine-readable instructions, which can be embodied in a program or software. The instructions may be stored in a memory location, such as the memory 1410. The instructions can be directed to the CPU 1405, which can subsequently program or otherwise configure the CPU 1405 to implement methods of the present disclosure. Examples of operations performed by the CPU 1405 can include fetch, decode, execute, and writeback.

The CPU 1405 can be part of a circuit, such as an integrated circuit. One or more other components of the system 1401 can be included in the circuit. In some cases, the circuit is an application specific integrated circuit (ASIC).

The storage unit 1415 can store files, such as drivers, libraries and saved programs. The storage unit 1415 can store user data, e.g., user preferences and user programs. The computer system 1401 in some cases can include one or more additional data storage units that are external to the computer system 1401, such as located on a remote server that is in communication with the computer system 1401 through an intranet or the Internet.

The computer system 1401 can communicate with one or more remote computer systems through the network 1430. For instance, the computer system 1401 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1401 via the network 1430.

Methods as described herein can be implemented by way of machine (e.g., computer processor) executable code stored on an electronic storage location of the computer system 1401, such as, for example, on the memory 1410 or electronic storage unit 1415. The machine executable or machine readable code can be provided in the form of software. During use, the code can be executed by the processor 1405. In some cases, the code can be retrieved from the storage unit 1415 and stored on the memory 1410 for ready access by the processor 1405. In some situations, the electronic storage unit 1415 can be precluded, and machine-executable instructions are stored on memory 1410.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1401, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1401 can include or be in communication with an electronic display 1435 that comprises a user interface (UI) 1440 for providing, for example, information on stool sample collection. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1405. The algorithm can, for example, determine parameters of stool collection (e.g., time course of sample collection), frequency of collection, analysis of the stool sample, etc.

### EXAMPLES

### Example 1—Automated Stool Processing

As described herein, a method for processing a stool sample may comprise providing a stool sample, optionally on a substrate, automatically processing the stool sample and/or substrate to generate a mixture, and extracting at least a portion, e.g., a biomolecule of the stool sample, from the mixture. In one example, a system as described herein, e.g., as shown in FIGS. 11A-11B, may be used to process a stool sample. In such an example, a stool sample may be collected on a substrate (e.g., toilet paper) and deposited into the system. The system may automatically perform one or more processing operations, including (i) providing a liquid (e.g., water), (ii) mixing, agitating, and/or homogenizing the stool sample, substrate, and liquid in a vessel, to generate a mixture, (iii) performing lysis of one or more cells contained in the mixture, to generate a lysed product and (iv) isolating one or more biomolecules, e.g., DNA, from the lysed product.

In some instances, such processing operations may be performed in one or more system components. For example, (i) and (ii) may be performed in a vessel, (iii) may be performed in a lysis unit, and (iv) may be performed in a storage unit. Alternatively, (i) and (ii) may be performed in a vessel, and (iii) and (iv) may be performed in a lysis unit. It will be appreciated that the different operations may be performed in different orders and in different system components.

In one example, (iv) may occur in the storage unit. For example, the lysed product may be provided into one or more wells of a multi-well unit (or other a sample container). Using a fluid handling unit comprised within the storage unit, a DNA-binding bead and a binding buffer may be provided to the one or more wells. Alternatively or in addition to, the bead and/or buffers may be provided in the sample container that is loaded into the storage unit. Any non-bound reagents and liquid may optionally be removed (e.g., aspirated). The one or more wells may be subjected to washing, and in some instances, elution. The sample container (e.g., multi-well unit) may be removable from the system and additional characterization may be performed. Further characterization may comprise sequencing or running one or more assays (e.g., immunoassays or other proteomic assays).

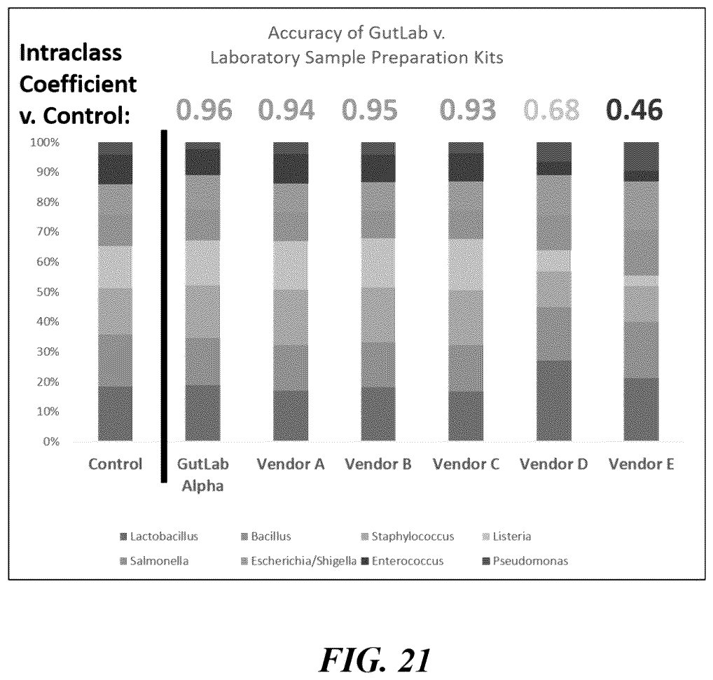

FIG. 21 shows example data from an automated method of processing a mock stool sample, as compared to conventional laboratory techniques. In such an example, a mock microbial community standard (Zymo Research) is processed using the automated methods and systems described herein (“GutLab Alpha”) and sequenced to determine the relative abundance of different bacterial strains (or genera) within the standard. The sequencing data obtained from the automated methods and systems described herein is compared to the sequencing data obtained from sample processing using conventional laboratory-grade kits that are commercially available (“Vendor A,” “Vendor B,” “Vendor C,” “Vendor D,” “Vendor E”). The columns graphs show, from left to right, the relative abundance of DNA isolated for the different bacterial strains (or genera) for a control group (as measured by direct sequencing of the standard), the methods and systems described herein (“GutLab Alpha”), and the different laboratory techniques (Vendors A-E). The accuracy of each measurement is measured by comparing an intraclass coefficient compared to the control. As can be seen in FIG. 21, the GutLab Alpha produces highly accurate characterization of the microbial community, as compared to the conventional laboratory standards (Vendors A-E). Altogether, these results indicate that the automated processing methods and systems described herein may be useful in accurately characterizing microbial populations.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A method for processing a stool sample, comprising:
(a) providing (i) a substrate comprising said stool sample and (ii) a liquid;
(b) processing said substrate, thereby generating a mixture comprising said liquid and portions of said substrate; and
(c) extracting at least a portion of said stool sample from said mixture, thereby generating a processed stool sample.

2.-23. (canceled)

24. A method for processing a stool sample, comprising:
(a) providing a substrate comprising said stool sample;
(b) agitating said substrate to yield a plurality of portions of said substrate, wherein a portion of said plurality of portions of said substrate comprises at least a portion of said stool sample; and
(c) extracting a biomolecule from said at least said portion of said stool sample of (b).

25.-44. (canceled)

45. A system for processing a stool sample provided on a substrate, comprising:
a vessel configured to process (i) said stool sample provided on said substrate and (ii) a liquid, to generate a mixture comprising said liquid and portions of said substrate; and
an extraction unit configured to extract at least a portion of said stool sample from said mixture to generate a processed stool sample.

46. The system of claim 45, wherein said extraction unit comprises a lysis unit configured to lyse one or more cells present in said mixture to generate a lysed product.

47. The system of claim 46, wherein said extraction unit further comprises a storage unit configured to extract said at least said portion of said sample from said lysed product.

48. The system of claim 47, wherein said storage unit is in fluidic communication with a reservoir comprising reagents for extracting a biomolecule from said at least said portion of said sample.

49. The system of claim 48, wherein said biomolecule is a cell, an organelle, a protein, a peptide, a lipid, a carbohydrate, or a nucleic acid molecule.

50. The system of claim 47, wherein said storage unit comprises a support for capturing a biomolecule from said at least said portion of said sample.

51. The system of claim 50, wherein said support is a bead.

52. The system of claim 51, wherein said bead is a magnetic bead.

53. The system of claim 45, further comprising a waste unit configured to store and/or expel waste from said vessel or said extraction unit.

54. The system of claim 45, further comprising a storage unit configured to store a biomolecule extracted from said processed stool sample.

55. The system of claim 54, wherein said storage unit comprises a moveable stage configured to couple to a sample container.

56. The system of claim 55, wherein said sample container is disposable.

57. The system of claim 45, further comprising a reservoir in fluidic communication with said extraction unit.

58. The system of claim 45, further comprising a metering unit configured to meter volumes of liquids provided to said extraction unit and/or said vessel.

59.-60. (canceled)

61. The system of claim 45, wherein said vessel comprises a chamber for processing said stool sample and said liquid.

62. The system of claim 45, wherein said vessel comprises a blade, propeller, or blender for mixing, agitating, or homogenizing said stool sample and said liquid.

63. The system of claim 45, wherein said extraction unit comprises a storage unit, wherein said storage unit is configured to couple to a sample container.

64. The system of claim 63, wherein said sample container is a multi-well plate.
